(19) United States
(12) Patent Application Publication
(10) Pub. No.: US 2020/0299761 A1
(43) Pub. Date: Sep. 24, 2020

(54) PRE-TRANSPLANT TCR CLONALITY ASSESSMENT TO PREDICT POST-LIVER TRANSPLANT SURVIVAL

(71) Applicant: THE METHODIST HOSPITAL SYSTEM, Houston, TX (US)

(72) Inventors: Xian Chang Li, Houston, TX (US); Rafik Mark Ghobrial, Houston, TX (US); Krupa Ramaprased Mysore, Houston, TX (US)

(21) Appl. No.: 16/087,896

(22) PCT Filed: Mar. 23, 2017
(86) PCT No.: PCT/US17/23756

§ 371 Date: Sep. 24, 2018

Related U.S. Application Data
(60) Provisional application No. 62/312,317, filed on Mar. 23, 2016.

(51) Int. Cl. C12Q 1/6869; C12N 15/10; G01N 33/50

(52) U.S. Cl. CPC C12Q 1/6869; C12N 15/1003; G01N 33/5094; A61B 2017/00969; G01N 2800/085; G01N 2800/26; G01N 2800/52

(57) ABSTRACT

Disclosed herein are methods for scoring a patient on a liver transplant list, methods of performing a liver transplant, methods of determining expected post-transplant mortality in a subject, and methods of determining expected sepsis. The disclosed methods can be used to avoid futile transplantation, avoid wasting organs, and promote efficient management of organ placement. These methods involve assaying a sample from the subject for T cell receptor (TCR) repertoire.

### CROSS REFERENCE TO RELATED APPLICATIONS

This application claims the benefit of priority to U.S. Provisional Application No. 62/312,317, filed Mar. 23, 2016, which is incorporated by reference herein in its entirety.

### BACKGROUND

Liver transplantation has become the definitive treatment for patients with end-stage liver disease (Ghobrial R M, et al. Ann Surg 2002 236(3):315-322). Since 1987, the rate of new registration to the United Network for Organ Sharing (UNOS) waiting list has far exceeded the growth of cadaveric liver donors. The increasing numbers of patients awaiting liver transplantation, coupled with a limited donor pool, has resulted in: (i) a large number of patients die on the waiting list without liver transplantation and (ii) a higher proportion of patients undergoing transplantation when critically ill. In 2000, the Department of Health and Human Services (DHHS), established the “final rule” as a regulatory framework for structure and operation of Organ Procurement and Transplantation Network. Such rule demands that organ allocation shall be in accordance with: (i) to allocate organs among transplant candidates in order of medical urgency status, but (ii) to avoid futile transplantation, to avoid wasting organs, and to promote efficient management of organ placement. However, some authors argued that liver transplantation of critically ill patients represents a futile effort, since liver transplantation of critically ill recipients results in lower survival than less urgent patients and that the final rule mandates two opposing demands (Ghobrial R M, et al. Ann Surg 2002 236 (3):315-322; Bronsther O, et al. JAMA 1994 271 (2):140-143; Markmann J F, et al. Ann Surg 1997 226 (4):408-420). Liver transplantation has, therefore, focused on the selection of patients, from the large pool of medically urgent patients, who will benefit the most from the transplant procedure. Thus, pre-transplant prediction of post-transplant survival has become the “holy grail” of liver transplantation.

Determination of medical urgency, to satisfy the first part of the final rule, has been challenging. Historically, liver organs were allocated first to patients in the intensive care units, followed by hospitalized and, finally, to patients who were at home (Bronsther O, et al. JAMA 1994 271 (2):140-143). Such a highly subjective allocation system was modified several times until the development of the more objective Model for End-stage Liver Disease (MELD) system (Desai N M, et al. Transplantation 2004 77 (1):99-106). The MELD score, which ranges between the lowest of 6 and highest of 40, is calculated from serum bilirubin, creatinine and international normalized ratio for prothrombin time (INR). The validity of the model was based on the c (concordance)-statistic (concordance to the area under the operating curve), which ranges from 0-1, with 1 being a perfect correlation and 0.5 the result of chance alone. MELD demonstrated the ability to predict 3-month mortality from liver disease with a c-statistic of 0.78-0.87. Less than a perfect model, the MELD was nevertheless adopted by the Organ Procurement and Transplantation Network (OPTN) for distribution of liver organs to patients. The highest priority is given to the patients with the highest MELD. Whereas MELD has a relatively high predictive value for death on the waiting list, it exhibits a much lower predictive ability for survival post-transplantation (Ghobrial R M, et al. Ann Surg 2002 236 (3): 315-322; Wiesner R H, et al. Liver Transpl 2001 7 (7):567-580; Brown Jr R S, et al. Liver Transpl 2002 8 (3): 278-284). Therefore, MELD provides poor prediction of post-transplant survival (Ghobrial R M, et al. Ann Surg 2002 236 (3):315-322; Wiesner R H, et al. Liver Transpl 2001 7 (7):567-580; Brown Jr R S, et al. Liver Transpl 2002 8 (3):278-284).

The current challenge, in order to satisfy the second portion of the final rule, is to adequately define post-transplant survival using pre-transplant characteristics. Several models that utilized clinical criteria were developed (Ghobrial R M, et al. Ann Surg 2002 236 (3):315-322; Wiesner R H, et al. Liver Transpl 2001 7 (7):567-580; Brown Jr R S, et al. Liver Transpl 2002 8 (3):278-284). However, these models use operative and donor parameters that are difficult to identify in the pre-transplant period because such parameters are not known until after transplantation is completed. In addition, the currently available clinical models exhibit a low c-statistic of 0.67-0.69 (Ghobrial R M, et al. Ann Surg 2002 236 (3): 315-322). To date, the approach taken by most clinicians to determine the futility of transplanting a critically-ill patient is empirical. Accordingly, some patients may be denied a life-saving liver transplant due subjective perceptions rather than objective criteria to determine the outcome of liver transplantation.

### SUMMARY

Disclosed herein is a method of scoring a subject on a liver transplant list, which can be used to avoid futile transplantation, avoid wasting organs, and promote efficient management of organ placement. The method involves obtaining a blood sample from the subject; wherein the blood sample comprises peripheral blood mononuclear cells; extracting DNA from the peripheral blood mononuclear cells; sequencing the DNA and identifying sequences coding a region of a T cell receptor; and determining T cell clonality from the identified sequences, thereby scoring the subject.

Also disclosed herein is an in vitro method for determining expected post-liver transplant mortality in a subject. The method involves assaying T cell clonality from a sample obtained from the subject prior to a liver transplantation procedure, wherein the expected post-liver transplant mortality of the subject is determined to be high when the T cell clonality is greater than 0.3. The method can additionally or alternatively involve determining the expected post-liver transplant mortality in a subject when their T cell clonality is within 5% of, or is less than, the T cell clonality of a healthy individual or the average T cell clonality of a population of healthy individuals.

Also, disclosed herein is a method of performing a liver transplant. The method involves identifying a subject having a T cell clonality of 0.3 or less, preferably 0.2 or less; and transplanting a liver in the subject. The method can additionally or alternatively involve identifying a subject when their T cell clonality is within 5% of, or is less than, the T cell clonality of a healthy individual or the average T cell clonality of a population of healthy individuals; and transplanting a liver in the subject.

Further disclosed herein is an in vitro method for determining expected sepsis risk in a subject. The method involves assaying T cell clonality from a sample obtained from the subject, wherein the expected sepsis risk of the subject is determined to be high when the T cell clonality is greater than 0.3 (or is determined to be low when the T cell clonality is 0.3 or less, preferably 0.2 or less). The method can additionally or alternatively involve determining a low expected sepsis risk in a subject when their T cell clonality is within 5% of, or is less than, the T cell clonality of a healthy individual or the average T cell clonality of a population of healthy individuals.

The disclosed methods can involve assaying a blood sample from the subject prior to organ transplantation for T-cell receptor (TCR) repertoire. In these methods, a high T-cell clonality in the sample, e.g., quantified by DEEP sequencing of the CDR3 region of the T-cell receptor Vβ chain, is an indication that the subject has a high risk of mortality within a year post-transplantation. Therefore, in some embodiments, the methods further comprises selecting the subject for transplantation if the TCR repertoire is diverse.

TCR loci undergo combinatorial rearrangement, generating a diverse immune receptor repertoire, which is vital for recognition of potential antigens. Multiplex PCR can be used with a mixture of primers targeting the rearranged variable and joining segments to capture receptor diversity. Most of the diversity in TCRs is contained in the complementary determining region 3 (CDR3) regions of the heterodimeric cell-surface receptors. The CDR3 regions are formed by rearrangements of variable and joining (VJ) gene segments for the α and γ chains and variable, diversity and joining (VDJ) gene segments for the β and δ chains. The V-J, V-D and D-J junctions are imperfect rearrangements, and can have both deletions and non-templated nucleotide insertions. In addition to the generation of a diverse set of antigen receptor molecules, the adaptive immune system functions in part by clonal expansion. Therefore, in some embodiments, TCR repertoire can be assayed by DEEP sequencing of the TCR complementarity-determining region 3 (CDR3) regions. In these embodiments, a T cell clonality of 0.3 or less (e.g., 0.2 or less) can depict a diverse TCR repertoire and therefore favorable patient outcomes.

The disclosed method can also involve scoring the subject for pre-transplant mortality risk, e.g., to allocate organs among transplant candidates in order of medical urgency status. The lung allocation score (LAS) for lung transplants combines predicted waiting list survival and post-transplant survival. However, debate continues over whether the LAS predicts post-transplant survival at 1 year or beyond (see Shafli et al 2014 Ann Thoracic Surg; Maxwell et al 2014 Am J Transplant) and infection is the leading cause of death after lung transplant (Valapour et al 2015, Am J Transplant). Additionally, for example, in some embodiments the transplant organ is liver. In these embodiments, the method can further involve scoring the subject for pre-transplant mortality risk using a Model for End-Stage Liver Disease (MELD) scoring system. MELD is the standard score that is computed and entered in UNOS for all patients listed for liver transplantation. Currently, UNOS does not allow use of any other scoring parameter. MELD score does not predict mortality after transplant. It is only used for organ allocation as a predictor of who has a greater likelihood of dying while waiting for a liver transplant. For example, in 2012, approximately 27% of patients on the waiting list were either too sick to transplant (6%) or died while waiting (21%). The average MELD at transplant was 22 across the US with wide variations in MELD across donor services areas/regions. In some embodiments, the method can further comprise selecting the subject for transplantation if the TCR repertoire demonstrates high clonality and the MELD score is high. For example, the subject can be selected for transplantation if they have a T cell clonality of 0.3, 0.25, 0.2, 0.15, 0.1 or less and a MELD score higher than the regional average (e.g., 22). In a specific example, the subject can be selected for transplantation if they have a T cell clonality of 0.3 or less (e.g., 0.2 or less) and a MELD score of 22 or more.

In some embodiments, the disclosed methods can be used with any organ transplant system where there is a risk of post-transplant mortality from infection, e.g., sepsis. Therefore, in some embodiments, the transplant organ is lung, heart, kidney, pancreas, bone marrow, or small intestine.

Also disclosed is a method for treating a subject with organ disease that involves scoring the subject pre-transplant for expected post-transplant mortality risk; assaying a sample from the subject prior to organ transplantation for the TCR repertoire to determine post-transplant mortality risk; and replacing the organ in the subject with a donor organ if the TCR repertoire shows high clonality and the MELD score is high. In some embodiments, the method comprises treating the subject with palliative care if the T cell clonality is high, e.g., greater than 0.3, 0.35, 0.4, 0.45, 0.5, 0.55, 0.6, 0.65, 0.7, 0.75, 0.8, 0.85, 0.9, or 1.0.

In some embodiments, the disclosed methods are based on the ability of TCR diversity to determine expected sepsis risk. Therefore, also disclosed is a method for determining expected sepsis risk in a subject that involves assaying a sample from the subject for T cell receptor (TCR) repertoire, wherein a T cell clonality in the sample of greater than 0.3 is an indication that the subject has a high risk of sepsis. In some embodiments, the subject is immunocompromised. In some embodiments, the subject is taking immunosuppressive drugs. In some cases, the subject is elderly. In some cases, the subject is in intensive care and/or on a ventilator. In some cases, the subject has chronic viral infections. In some cases, the subject is on dialysis. In some cases, the subject has prolonged hospitalization. In some cases, the subject has two or more indwelling catheters.

In some embodiments, the sepsis comprises surgical sepsis, and the sample is obtained prior to a surgery. For example, the surgery can comprise organ transplantation. In some embodiments, underlying liver disease can compromise transplantation of organs other than liver. The elderly and other immune compromised patients, patients requiring prolonged hospitalization, patients with critical care needs requiring mechanical ventilation support, dialysis, or those having multiple indwelling catheters are also at increased risk.

In some cases, the methods involves selecting a non-surgical treatment option for the subject if high T cell clonality in the sample is detected. In some cases, the methods involves administering antibiotics to the subject after the surgery if high T cell clonality in the sample is detected. In some cases, the method involves identifying the root cause of the high clonality to restore normal TCR repertoire.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects described below.

FIGS. 1A to 1C show T-cell clonality in liver transplant patients who survived or died during the first year post-transplant (FIG. 1A) and show comparative receiver operating characteristic (ROC) curves for Model for End-stage Liver Disease (MELD) (FIG. 1B) or T-cell clonality (FIG. 1C).

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

As disclosed herein, T-cell clonality is a pre-transplant predictor for post-transplant survival. In some embodiments, this is due to its ability to predict sepsis, e.g. following surgical procedures.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “sample from a subject” as used herein refers to a tissue (e.g., tissue biopsy), organ, cell (including a cell maintained in culture), cell lysate (or lysate fraction), cellular material, or body fluid from a subject, so long as it contains T-cells or DNA from T-cells. For example, the sample can comprise peripheral blood mononuclear cells (PBMCs).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

The term “DEEP sequencing” refers to sequencing a genomic region multiple times, sometimes hundreds or even thousands of times.

The term “organ” as used herein refers to a structure of bodily tissue in mammal such as a human being wherein the tissue structure as a whole is specialized to perform a particular body function. Organs that are transplanted within the meaning of the present methods include skin, cornea, heart, lung, kidney, liver and pancreas. Solid organs include the heart, lung, kidney, liver, and pancreas.

The term “transplant” as used herein refers to any organ or body tissue that has been transferred from its site of origin to a recipient site. Specifically in an allograft transplant procedure, the site of origin of the transplant is in a donor individual and the recipient site is in another, recipient individual.

### T-Cell Clonality Assay

At a molecular level, the TCR is a heterodimer consisting of an α chain and a β chain. Structurally, each chain has a variable region (V region), which allows binding to diverse peptide antigens, and a constant region (C region). Extensive variations at the V region are generated through somatic recombination of variable (V), diversity (D), and joining (J) gene segments of the TCR α and β chains during T-cell development. The V region of the β chain is the most polymorphic, and gives rise to the most diversity. In humans, there are 54 V genes and 13 J genes, and any one of the V genes can pair with any one of 13 J genes to generate an extremely diverse TCR repertoire. Within the variable region of each TCR, there are 3 Complementarity-Determining Regions (CDR), and the CDR3 region is in direct contact with peptide antigens that are presented by the MHC-peptide complex and responsible for antigen binding, and therefore, CDR3 gives rise to the highest degree of diversity (Robins H S, et al. Blood 2009 114 (19):4099-4107). The diversity of the CDR3 region makes each CDR3 nucleotide sequence unique in individual T-cell clones. Based on the V-J usage in the CDR3 region, the new next generation DEEP sequencing technology (NextGen DEEP sequencing) provides a powerful platform that allows sequencing of the CDR3 region of the β chain in the entire TCR repertoire, thus allowing identification of individual T-cell clones and repertoire diversity in any given individual (Miconnet I. Curr Opin HIV AIDS 2012 7 (1):64-70). It should be noted that the composition and identity of individual T-cell clones vary considerably among individuals in the general population due to differences in vaccination history, frequency and nature of infections, history of immune activation, and age, etc.

In some cases, the method uses IMMUNOSEQ™ technology (Adaptive Biotechnologies), which allows ultra-DEEP sequencing of the TCRb CDR3 region and reveals the clonal composition of T cell populations. Briefly, the basic principle is a multiplexed PCR method that amplifies all possible rearranged genomic TCR β sequences in any given individual using 52 forward primers, each specific to a specific TCR Vβ segment, and 13 reverse primers, each specific to a specific TCR Jβ segment. High throughput reads of 60-bp length can be obtained using the Illumina HiSeq System. The raw HiSeq sequences can be processed to generate private and shard sequence database.

Clonality can be a measure equal to the inverse of the normalized Shannon entropy of all productive clones in the sample. Primary measure of entropy is calculated by summing the frequency of each clone times the log (base 2) of the same frequency over all productive reads in a sample. When this value is normalized based on the total number of productive unique sequences and subtracted from 1, a related measure, ‘clonality’, results.

Values for clonality range from 0 to 1. Values near 1 represent samples with one or a few predominant clones (monoclonal or oligoclonal samples) dominating the observed repertoire. Clonality values near 0 represent more polyclonal samples. In the methods disclosed herein, a clonality of 0.3 or less can be used to indicate a diverse T cell receptor repertoire, nominate a subject for transplant, indicate a low post-transplant mortality, and/or indicate a low risk of sepsis. T cell clonality of 0.2 or less can also be used, e.g., 0.20, 0.19, 0.18, 0.17, 0.16, 0.15, 0.14, 0.13, 0.12, 0.11, 0.10, 0.09 or less.

In some embodiments, the T cell clonality of a subject can be compared to the T cell clonality of a single healthy individual or the average T cell clonality of a population of healthy individuals determined by the same methods used to determine the subject's T cell clonality. The healthy individual or population of healthy individuals can share one or more factors with the subject chosen from age, gender, race, geographic location, socioeconomic status, history of alcohol consumption, and history of drug use. Thus, the disclosed methods can include a step of obtaining a T cell clonality of a healthy individual or average T cell clonality of a population of healthy individuals sharing one or more of these factors with the subject. The disclosed methods can also include the step of comparing the subject's T cell clonality with the T cell clonality of the healthy individual or average T cell clonality of the population of healthy individuals. In certain examples, the subject can be nominated for transplant when their T cell clonality is within 5% of the T cell clonality of a healthy individual or average T cell clonality of a population of healthy individuals. In certain examples, the subject can be nominated for transplant when their T cell clonality is lower than the T cell clonality of a healthy individual or average T cell clonality of a population of healthy individuals. Further, a subject's T cell clonality that is within 5%, or is less than, the T cell clonality of a healthy individual or average T cell clonality of a population of healthy individuals can be used to indicate a low post-transplant mortality and low risk of sepsis.

A diversity index can be calculated based on the Simpson index of diversity (D) where nᵢ is the total number of amino acid sequences belonging to type i, and N is the total number of sequences in the dataset for each individual (the formula inserted here).

$$D = 1 - \frac{\sum ni ( ni - 1 )}{N ( N - 1 )}$$

### Surgical Procedures/Sepsis

People undergoing general surgery have a 10 times greater risk of dying of sepsis and septic shock than from pulmonary embolism or myocardial infarction (MI), data from a national registry suggest. Any type of surgery exposes the subject's body to infection and a fair number of complications, many of which could develop into sepsis. The most common cause of sepsis after surgery is infection. This could be infection of the incision, where the surgeon opened to perform the procedure, or an infection that develops after the surgery, such as pneumonia or urinary tract infection (UTI).

Factors increasing the risk for sepsis or septic shock included older age, the need for emergency versus elective surgery, and comorbidity. Once sepsis sets in, if left untreated, it can progress to septic shock and death. Worldwide, one-third of people who develop sepsis die. Many who do survive are left with life-changing effects, such as post-traumatic stress disorder (PTSD), chronic pain and fatigue, and organ dysfunction (don't work properly) and/or amputations.

Solid organ transplant recipients require lifetime immunosuppression and are highly susceptible to opportunist and non-opportunistic infections. Sepsis is a serious post-transplant complication.

Sepsis can be simply defined as a spectrum of clinical conditions caused by the immune response of a patient to infection that is characterized by systemic inflammation and coagulation. It includes the full range of response from systemic inflammatory response syndrome (SIRS) to organ dysfunction to multiple organ failure and ultimately death. The American College of Chest Physicians and the Society of Critical Care Medicine developed the following definitions to clarify the terminology used to describe the spectrum of disease that results from severe infection. The basis of sepsis is the presence of infection and the subsequent physiologic alterations in response to that infection, namely, the activation of the inflammatory cascade. Systemic inflammatory response syndrome (SIRS) is a term used to define this clinical condition and it is considered present if abnormalities in two of the following four clinical parameters exist: (1) body temperature, (2) heart rate, (3) respiratory rate, and (4) peripheral leukocyte count. Sepsis is defined as the presence of SIRS in the setting of infection. Severe sepsis is defined as sepsis with evidence of end-organ dysfunction as a result of hypoperfusion. Septic shock is defined as sepsis with persistent hypotension despite fluid resuscitation and resulting tissue hypoperfusion. Bacteremia is defined as the presence of viable bacteria within the liquid component of blood. Bacteremia may be primary (without an identifiable focus of infection) or, more often, secondary (with an intravascular or extravascular focus of infection). While sepsis is commonly associated with bacterial infection, bacteremia is not a necessary ingredient in the activation of the systemic inflammatory response that results in severe sepsis. In fact, fewer than 50% of cases of sepsis are associated with bacteremia and severe sepsis or septic shock may develop in patients that undergo SIRS due to trauma, severe burns and other inflammatory stimuli wherein no infection can be detected. Patients with septic shock may have a biphasic immunological response. Initially, they manifest an overwhelming inflammatory response to the infection.

The time window for interventions is short and treatment must promptly control the source of infection and restore hemodynamic homeostasis. There is a continuum of clinical manifestations from SIRS to sepsis to severe sepsis to septic shock to Multiple Organ Dysfunction Syndrome (MODS). The first attempts to combat inflammation in patients with septic shock relied on non-selective drugs, i.e., high dose corticosteroids (D. Annane et al., BMJ 2004; 329:480) and non-steroidal inflammatory drugs (G. R. Bernard, N. Engl. J. Med. 1997; 336:912-918). These drugs failed to improve survival. Monoclonal antibodies (HA-IA, E5) targeting Mucopolysaccharide (LPS) were also tested, but proved ineffective because of their weak biological activity (E. J. Ziegler et al., N. Engl. J. Med. 1991; 324:429-436). Second-generation drugs for septic shock blindly and systemically block one factor in the inflammatory cascade, for instance, TNF-α, interleukin-1, platelet-activating factor, adhesion molecules or NO synthase.

The risk of post-transplant mortality and/or sepsis can be calculated by assaying a sample from the subject prior to organ transplantation for T cell receptor (TCR) repertoire. The clonality of the TCR repertoire is determined, e.g., where a highly clonal repertoire (or low diversity) is indicative of a higher risk of sepsis and/or post-transplant mortality.

A number of embodiments of the invention have been described. Nevertheless, it will be understood that various modifications may be made without departing from the spirit and scope of the invention. Accordingly, other embodiments are within the scope of the following claims.

### EXAMPLES

Liver transplantation is often the only choice of treatment for patients with end-stage liver failure. This procedure has brought hope to many patients suffering from liver diseases, and an overwhelming majority of them experience excellent quality of life following liver transplantation (Sullivan K M, et al. Liver Transpl 2014 20 (6):649-654). There are many diseases that eventually result in liver failure, which include cancer, hepatitis viruses, alcohol, and poisoning, so the patients represent a diverse cohort with very different primary diseases to begin with. A common feature among these patients is that they have to take immunosuppression drugs for life to prevent rejection of the liver transplant by the immune system. On the other hand, the immune system plays an essential role in fending off infections, and non-specific suppression will render patients vulnerable to infectious complications (Fishman J A. Cold Spring Harb Perspect Med 2013 3 (10):a015669).

Immunosuppression drugs broadly suppress the immune system and compromise immune responses to pathogens. Generally speaking, for patients with liver transplant, the one-year survival is around 90% in the US, which is considered excellent. However, ˜10% of patients die in the first year due to a variety of reasons, and most of them with a well-functioning liver graft (non-surgical death), and a substantial number of them die of infectious complications. Considering that there are approximately 6,000 liver transplants per year in the US, non-surgical death accounts for over 600 liver transplants. This is a significant number, and means to prevent such futile transplants can have significant impact in the field. To date there is no reliable pre-transplant markers to inform transplant physicians to avoid patient death after liver transplantation.

The disclosed methods address the issue of futile liver transplants from a new perspective. The disclosed methods focus on the entirety of patients' T-cell repertoire, which is the target of immunosuppression drugs as well as the effector of immune protection, and assess the entire spectrum of the T-cell receptor (TCR) diversity. Through high throughput DEEP sequencing, the clonality of a T-cell repertoire for individual patients was map out. As the T-cell repertoire is composed of millions of T-cell clones, and each T-cell clone is equipped with a unique TCR recognizing a specific antigen (Davis M M and Bjorkman P J. Nature 1988 334 (6181):395-402), the more diverse the TCR, the more clones in the T-cell repertoire, and the better the protection against pathogens. Conversely, the less diverse the TCR, the less effective in protection. This information can be obtained before liver transplantation. One of the common causes of a reduced TCR diversity (increased TCR clonality) is unbalanced expansion of a few dominant clones in the repertoire, resulting in a highly skewed T-cell repertoire.

A unique pre-transplant TCR signature was identified that is strongly correlated with patient death after liver transplantation, which is of great clinical significance. Peripheral blood mononuclear cells (PBMCs) were collected 24 hours pre transplantation from 14 subjects undergoing liver transplant for end-stage liver disease using Ficoll-Paque centrifugation method. Samples were analyzed in this pilot phase 1 diagnostic study using DEEP sequencing for the CDR3 region of TCRβ, and variability at the CDR3 region was used as a readout for T-cell clonality. Total genomic DNA was extracted from PBMC and TCRβ chain sequencing was performed at Adaptive Biotechnologies (Seattle, Wash.). A multiplex PCR system was used to amplify the rearranged CDR3β sequences from DNA using specific primers. The 87-base pair fragment identified the VDJ regions spanning each unique CDR3β. This is a quantitative assay utilizing a complete synthetic repertoire of TCRs to establish an amplification baseline and adjust the assay to correct for primer bias. In addition, bar-coded, spiked-in synthetic templates were used to measure sequencing coverage and residual PCR bias.

Bioinformatics analysis of all CDR3 sequences can be performed on the sequencing data using algorithms developed by Adaptive Biotechnologies on the ImmunoSEQ analyzer toolset. The sequencing data also determines the number and sequence of productive unique Vβ and Jβ genes in each sample, and thus mapping the entire T-cell repertoire. The nucleotide sequences can be used as an identifier for a particular T-cell clone across different samples and can be quantitatively assayed in the same patient to track clonal expansion or contraction of the T-cell repertoire. This analysis traces the TCR gene rearrangements and can track productive sequences acting as a fingerprint of each TCR and, in turn, each T lymphocyte. In general, calculated TCR clonality varies from 0 to 1 corresponding to a range of polyclonal to oligoclonal samples; the greater the number, the less diversity in the TCR repertoire, with 1 being no diversity (meaning the entire T-cell repertoire has 1 clone). It also helps in determination of the degree of clone sharing between samples, the frequency of clonal sequences and the diversity of TCRβ. In addition, the sequence analyzer also gives detailed information about the amino acid sequences of CDR3, which may allow future identification of specific antigens that stimulate such T-cell clones.

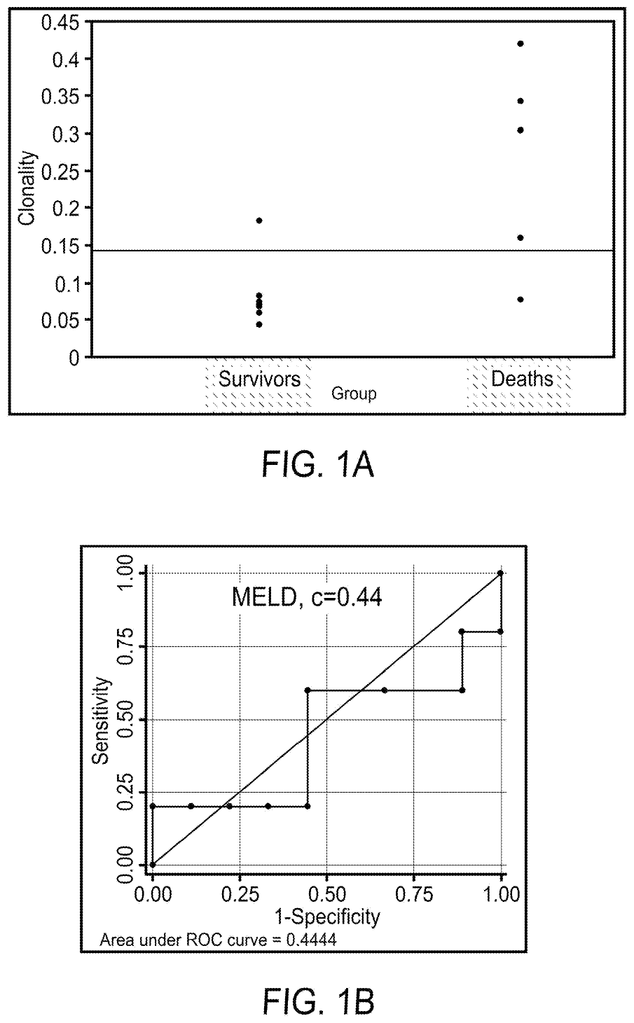

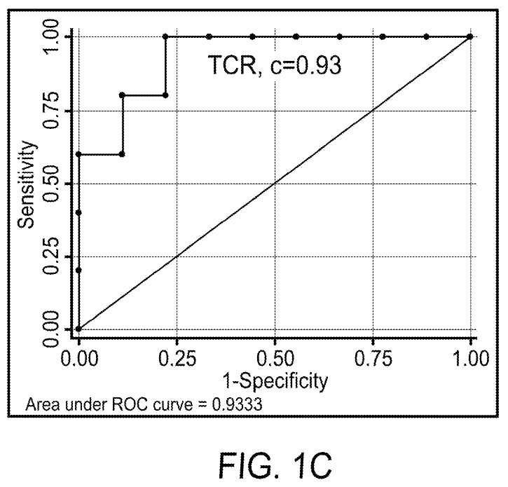

This 14-patient cohort included 9 recipients who were alive at one-year post-transplant (Survivors) and 5 liver transplant recipients who died within the first year after LT at Houston Methodist Hospital due to non-surgical reasons (3 to sepsis, 1 to cancer and 1 to GVHD; Deaths). Age was similar in both groups: 53±15 vs 58±6 y, p=NS. Similarly, pre-transplant MELD scores were similar (35±4 vs 32±12, p=NS). Additionally, there was no difference in all other clinical parameters between both groups. The only difference between Survivors versus Deaths was the pre-transplant T-cell clonality (0.075±0.042 vs 0.26±0.13, p=0.03; FIG. 1A). Additionally, the frequency of the top clones for each patient and the nucleotide sequence was analyzed. This showed that the patients with poor outcomes had the highest clonality and high frequency of a single clone of TCRβ. Oligoclonal T-cell expansion was associated with variable magnitude of skewing of the TCR repertoire. The predictive value of pre transplant clonality and MELD was further analyzed for post-transplant survival using the c-statistic. The pre-transplant MELD score appeared to predict outcomes post-transplant with a c-statistic of 0.444, consistent with poor prediction of post-transplant survival (FIG. 1B). In contrast, the ROC curve of pre transplant T-cell clonality was 0.933, suggestive of a potentially strong predictor of post-transplant outcomes (FIG. 1C).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of scoring a subject on a liver transplant list, comprising:
(a) obtaining a blood sample from the subject; wherein the blood sample comprises peripheral blood mononuclear cells;
(b) extracting DNA from the peripheral blood mononuclear cells;
(c) sequencing the DNA and identifying sequences coding a region of a T cell receptor; and
(d) determining T cell clonality from the identified sequences, thereby scoring the subject.

2. (canceled)

3. The method of claim 1, wherein the region of the T cell receptor is a beta chain, a complementarity-determining region 3, a variable region or a joining region.

4. (canceled)

5. (canceled)

6. The method of claim 1, further comprising nominating the subject for a liver transplant when the T cell clonality is 0.3 or less.

7. (canceled)

8. The method of claim 1, wherein sequencing the DNA is by DEEP sequencing.

9. (canceled)

10. The method of claim 1, further comprising determining T cell clonality of a healthy individual or average T cell clonality of a population of healthy individuals.

11. The method of claim 10, further comprising scoring the subject when the T cell clonality of the subject is within 5% of, or is lower than, the T cell clonality of the healthy individual or the average T cell clonality of the population of healthy individuals.

12. (canceled)

13. An in vitro method for determining expected post-liver transplant mortality in a subject, comprising: assaying T cell clonality from a sample obtained from the subject prior to a liver transplantation procedure, wherein the expected post-liver transplant mortality of the subject is determined to be high when the T cell clonality is greater than 0.3 or when the T cell clonality is within 5% of, or is less than, the T cell clonality of a healthy individual or the average T cell clonality of a population of healthy individuals.

14. The method of claim 13, further comprising selecting the subject for liver transplantation when the T cell clonality is 0.3 or less.

15. (canceled)

16. The method of claim 13, further comprising scoring the subject for pre-transplant mortality risk using a Model for End-Stage Liver Disease scoring system.

17. The method of claim 16, further comprising selecting the subject for transplantation when the TCR clonality is 0.3 or less and the Model for End-Stage Liver Disease score is 22 or more.

18. (canceled)

19. The method of any one of claims 13-18, wherein the sample is assayed by sequencing a region coding a beta chain, a complementarity-determining region 3, a variable region and/or a joining region of a T cell receptor.

20. (canceled)

21. (canceled)

22. (canceled)

23. The method of claim 13, wherein the sample comprises peripheral blood mononuclear cells.

24. A method of performing a liver transplant, comprising:
(a) identifying a subject having a T cell clonality of 0.3 or less or within 5% of, or less than, the T cell clonality of a healthy individual or the average T cell clonality of a population of healthy individuals; and
(b) transplanting a liver in the subject.

25. The method of claim 24, wherein the T cell clonality is determined by:
(a) obtaining a blood sample from the subject; wherein the blood sample comprises peripheral blood mononuclear cells;
(b) extracting DNA from the peripheral blood mononuclear cells;
(c) sequencing the DNA and identifying sequences coding a region of a T cell receptor; and
(d) determining T cell clonality from the identified sequences.

26. (canceled)

27. The method of claim 24, wherein the region of the T cell receptor is a beta chain, a complementarity-determining region 3, a variable region or a joining region.

28. (canceled)

29. (canceled)

30. The method of claim 25, further comprising scoring the subject for pre-transplant mortality risk using a Model for End-Stage Liver Disease scoring system.

31. The method of claim 30, further comprising identifying the subject with a Model for End-Stage Liver Disease score of 22 or more.

32. An in vitro method for determining expected sepsis risk in a subject, comprising: assaying T cell clonality from a sample obtained from the subject, wherein the expected sepsis risk of the subject is determined to be high when the T cell clonality is greater than 0.3 and the expected sepsis risk of the subject is determined to be low when the T cell clonality is within 5% of, or is less than, the T cell clonality of a healthy individual or the average T cell clonality of a population of healthy individuals.

33. (canceled)

34. The method of claim 32, wherein the sample is assayed by sequencing a region coding a beta chain, a complementarity-determining region 3, a variable region and/or a joining region of a T cell receptor.

35. (canceled)

36. (canceled)

37. (canceled)

38. The method of claim 32, wherein the sample comprises peripheral blood mononuclear cells.

39. (canceled)

40. The method of claim 32, wherein the sepsis comprises surgical sepsis, and wherein the sample is obtained prior to a surgery.

41. The method of claim 32, further comprising administering antibiotics to the subject.

42. (canceled)

43. (canceled)

44. (canceled)
